(19) United States
(12) Patent Application Publication
(10) Pub. No.: US 2017/0112443 A1
(43) Pub. Date: Apr. 27, 2017

(54) SENSOR APPARATUS AND MEASUREMENT APPARATUS HAVING THE SAME

(71) Applicant: National Chiao Tung University, Hsinchu (TW)

(72) Inventors: Chin-Teng Lin, Hsinchu (TW); Yi-Hsin Yu, Hsinchu (TW)

(21) Appl. No.: 15/297,927

(22) Filed: Oct. 19, 2016

(30) Foreign Application Priority Data
Oct. 21, 2015 (TW) 104134503

(51) Int. Cl. A61B 5/00; A61B 5/0478; A61B 5/01

(52) U.S. Cl. CPC A61B 5/6834; A61B 5/01; A61B 5/0478; A61B 5/6814; A61B 2562/0247; A61B 2562/0209; A61B 2562/222

(57) ABSTRACT

A sensor apparatus is provided, including: a hollow cylindrical sleeve having a first side and a second side disposed on two end openings thereof, respectively; an electrode disposed on the first side of the sleeve for measuring a signal; a conductive line electrically connected to the electrode for transmitting the signal measured by the electrode; and a siphoning portion having a suction port connected and tightly sealed with the second side of the sleeve and an outlet port for discharging gas from the sleeve, thus creating a vacuum effect to enable the electrode to be sucked to the head of a subject. Therefore, the present invention avoids the conventional drawback of discomfort of the subject caused by the use of a conductive material.

### BACKGROUND OF THE INVENTION

1. Technical Field

The present invention relates to sensor apparatuses, and, more particularly, to a sensor apparatus having a siphoning portion and a measurement apparatus having a plurality of the sensor apparatuses.

2. Description of Related Art

Human brain can think and judge. During the thinking or judging process, neurons of the brain continuously generate electrical impulses for processing information. The electrical impulses are called brainwaves, which can be monitored through an instrument. Brainwave signals indicate the activity of the brain and can be measured and recorded for analysis.

To measure brainwaves, a brainwave measurement apparatus is usually attached to a subject to record brainwave signals during a certain time period for analysis. Before the measurement, the scalp is cleaned with alcohol and electrode wires are fastened to the head with a conductive adhesive. However, long time use of the conductive adhesive may cause the subject to feel uncomfortable and even cause skin irritation, thus reducing the willingness to use the apparatus. According to another brainwave measurement method, conductive metal is directly pressed against the scalp to measure brainwaves, which, however, leads to a poor measurement effect and causes the subject to feel pressure and discomfort.

Therefore, how to overcome the above-described drawbacks has become critical. Particularly, there is a need to find a dry-type brainwave measurement method so as to allow electrodes to be suitably attached to the scalp of a subject without being over-pressed or causing any poor contact.

### SUMMARY

In view of the above-described drawbacks, the present invention provides a sensor apparatus, which comprises: a hollow cylindrical sleeve having a first side and a second side disposed on two end openings thereof, respectively; an electrode disposed on the first side of the sleeve for measuring a signal; a conductive line electrically connected to the electrode for transmitting the signal measured by the electrode; and a siphoning portion having a suction port connected and tightly sealed with the second side of the sleeve and an outlet port for discharging gas from the sleeve.

In an embodiment, the sleeve is made of silicone.

In an embodiment, the sleeve has a diameter of from 0.5 to 2 cm.

In an embodiment, the signal relates to temperature, pressure or current.

In an embodiment, after the gas is discharged from the sleeve, an internal pressure formed in the sleeve enables the sensor apparatus to be sucked to the skin of a person.

The present invention further provides a measurement apparatus, which comprises a plurality of sensor apparatuses and a pumping unit. Each of the sensor apparatuses comprises: a hollow cylindrical sleeve having a first side and a second side disposed on two end openings thereof, respectively; an electrode disposed on the first side of the sleeve for measuring a signal; a conductive line electrically connected to the electrode for transmitting the signal measured by the electrode; and a siphoning portion having a suction port connected and tightly sealed with the second side of the sleeve and an outlet port for discharging gas from the sleeve. The pumping unit comprises a collection portion having a plurality of communication ports communicating with the outlet ports of the sensor apparatuses, respectively, allowing the gas from the sleeves of the sensor apparatuses to be collected in the collection portion and discharged through the pumping unit.

In an embodiment, the measurement apparatus further comprises a signal unit connected to the conductive lines of the sensor apparatuses for collecting the signals measured by the electrodes of the sensor apparatuses.

In an embodiment, the signal unit integrates the signals into single-channel measurement signals.

In an embodiment, at least one of the sleeves is made of silicone.

In an embodiment, at least one of the sleeves has a diameter of from 0.5 to 2 cm.

According to the sensor apparatus of the present invention, gas is discharged from the sleeve through the siphoning portion so as to create a vacuum effect to enable the electrode to be sucked to the head of a subject, thus achieving a preferred attachment effect. Also, the present invention overcomes the conventional drawback of discomfort of the subject caused by the use of a conductive material such as a conductive adhesive or metal.

In addition, according to the measurement apparatus of the present invention, a plurality of sensor apparatuses can be sucked to the head of the subject at the same time so as to increase the contact area and obtain more brainwave signals.

### BRIEF DESCRIPTION OF DRAWINGS

FIG. 1 is a schematic exploded view of a sensor apparatus according to the present invention;

FIG. 2 is a schematic assembly view of the sensor apparatus according to the present invention;

FIG. 3 is a schematic assembly view of a measurement apparatus having a plurality of sensor apparatuses according to the present invention; and

FIG. 4 is a schematic view showing application of the measurement apparatus according to the present invention.

### DETAILED DESCRIPTION

The following illustrative embodiments are provided to illustrate the present invention, these and other advantages and effects can be apparent to those in the art after reading this specification. It should be noted that all the drawings are not intended to limit the present invention. Various modifications and variations can be made without departing from the spirit of the present invention.

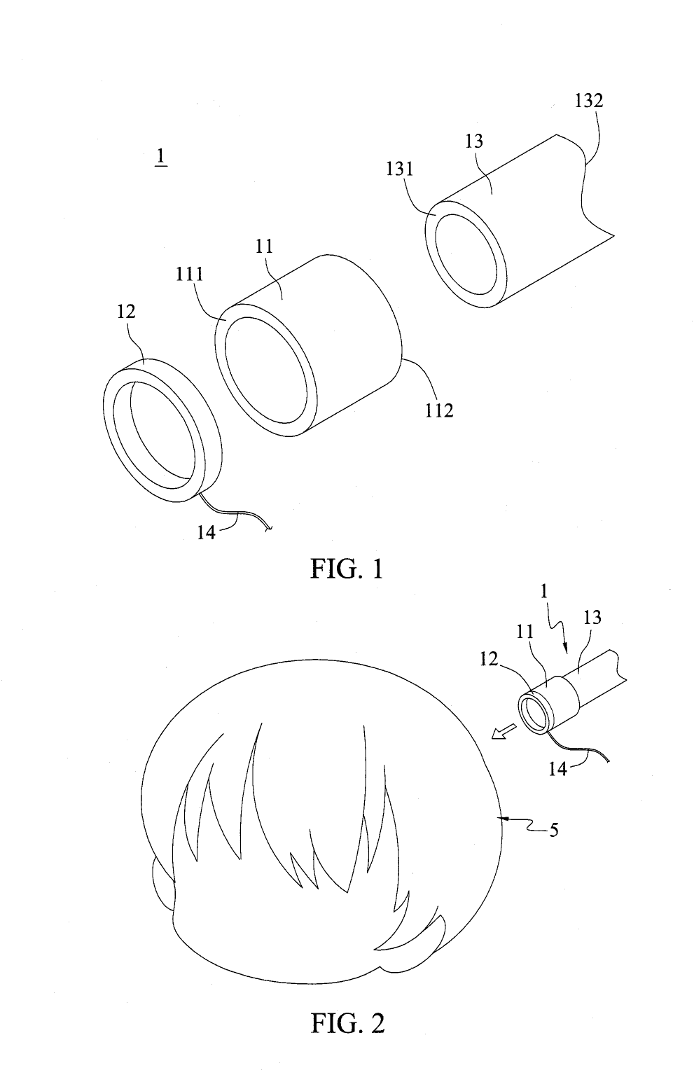

FIG. 1 is a schematic exploded view of a sensor apparatus 1 according to the present invention. The sensor apparatus 1 has a hollow cylindrical sleeve 11, an electrode 12, a siphoning portion 13 and a conductive line 14.

The sleeve 11 has a first side 111 and a second side 112 disposed on two end openings thereof, respectively. The first side 111 of the sleeve 11 is positioned close to the scalp of a subject and connected to the electrode 12, and the second side 112 of the sleeve 11 is positioned close to a pumping device (not shown).

The electrode 12 is disposed on the first side 111 of the sleeve 11 for measuring a signal, and the conductive line 14 is electrically connected to the electrode 12 for transmitting the signal measured by the electrode 12 to a back-end storage device (not shown) for storage and analysis.

The siphoning portion 13 has a suction port 131 and an outlet port 132, and the suction port 131 is connected and tightly sealed with the second side 112 of the sleeve 11. As such, gas can be pumped out of the sleeve 11 and discharged through the outlet port 132 of the siphoning portion 13.

The sleeve 11 can be made of an anti-static and anti-electromagnetic material. In an embodiment, the sleeve 11 of the sensor apparatus 1 is made of silicone.

In an embodiment, the sleeve 11 of the sensor apparatus 1 has a diameter of from 0.5 to 2 cm, preferably from 0.5 to 1 cm.

In an embodiment, the conductive line 14 of the sensor apparatus 1 is used for transmitting the signal measured by the electrode 12. The signal measured by the electrode 12 can relate to temperature, pressure or current. For example, the sensor apparatus 1 according to the present invention can be used for measuring brainwave signals.

After the gas is pumped out of the sleeve 11 of the sensor apparatus 1, the internal pressure formed in the sleeve 11 enables the sensor apparatus 1 to be securely sucked to the scalp or skin of the subject, thus achieving a preferred attachment effect without causing discomfort of the subject.

In the sensor apparatus 1, the gas is pumped out of the sleeve 11 and discharged through the siphoning portion 13 so as to enable the electrode 12 at the front end of the sleeve 11 to be suitably attached to the scalp of the subject. Therefore, the present invention achieves a preferred attachment effect without the need to press the electrode 12 hard against the scalp of the subject.

FIG. 2 is a schematic assembly view of the sensor apparatus 1 according to the present invention. To measure brainwave signals of the subject, the electrode 12 of the sensor apparatus 1 is attached to the scalp 5 of the subject, and a pumping device is used to pump gas out of the sleeve 11 through the siphoning portion 13. As such, a low pressure is formed in the sleeve 11 to enable the electrode 12 to be tightly sucked to the scalp 5 of the subject. Therefore, the present invention overcomes the conventional drawback of discomfort of the subject caused by the use of a conductive material such as a conductive adhesive or metal. Further, the conductive line 14 electrically connected to the electrode 12 can transmit the signal measured by the electrode 12.

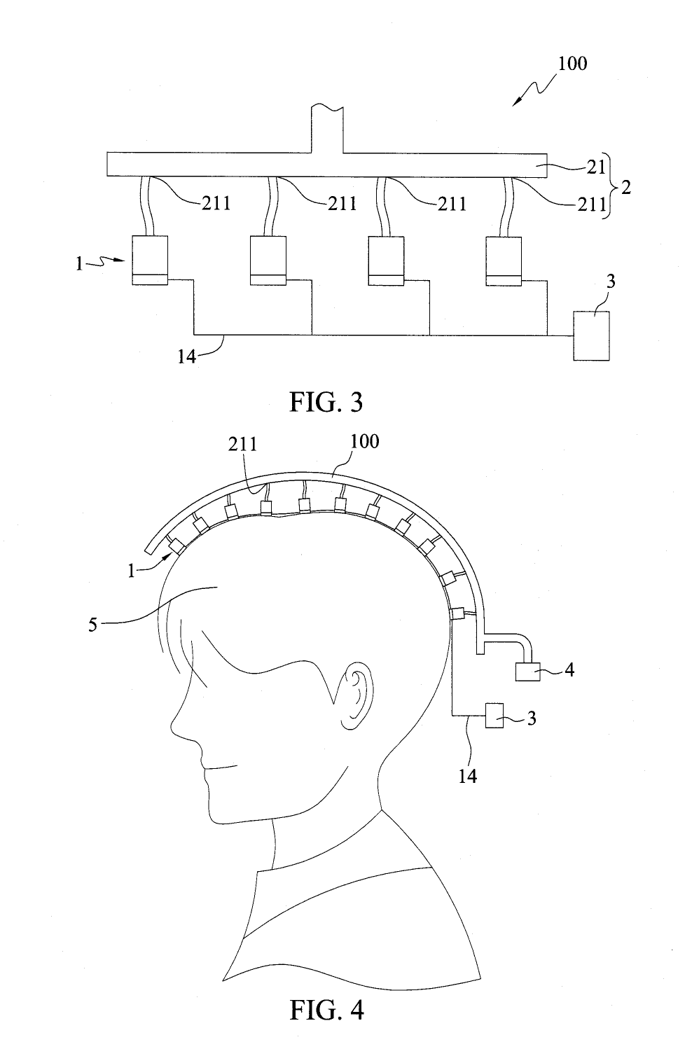

FIG. 3 is a schematic assembly view of a measurement apparatus 100 having a plurality of sensor apparatuses according to the present invention. The measurement apparatus 100 has a plurality of sensor apparatuses 1 of FIG. 1, a pumping unit 2 for pumping gas out of the sensor apparatuses 1, and a signal unit 3 for collecting measured signals.

Referring to FIGS. 1 and 3, the pumping unit 2 has a collection portion 21 having a plurality of communication ports 211 communicating with the outlet ports 132 of the sensor apparatuses 1, respectively. As such, during a pumping process, gas from the sleeves 11 of the sensor apparatuses 1 passes through the corresponding outlet ports 132 of the siphoning portions 13 of the sensor apparatuses 1 and is collected in the collection portion 21 and then discharged by the pumping unit 2.

In an embodiment, the measurement apparatus 100 is provided with a plurality of sensor apparatuses 1, and in operation, all the sensor apparatuses 1 will be sucked to the skin of the subject through collective gas pumping. In an embodiment, the outlet ports 132 of the siphoning portions 13 of the sensor apparatuses 1 are connected and tightly sealed with the communication ports 211 of the pumping unit 2, respectively, thus allowing gas from the sleeves 11 of the sensor apparatuses 1 to be collected in the collection portion 21 of the pumping unit 2. As such, a low pressure is formed in the sleeves 11 of the sensor apparatuses 1 so as to enable the electrodes 12 of the sensor apparatuses 1 to be tightly sucked to the skin of the subject.

Further, the signal unit 3 is connected to the conductive lines 14 of the sensor apparatuses 1 to collect the signals measured by the electrodes 12. The collected signals are then analyzed, and useful signals are thus retained.

In another embodiment, the signal unit 3 integrates the signals measured by the sensor apparatuses 1 into single-channel measurement signals. In an embodiment, to measure brainwave signals of the subject, a plurality of sensor apparatuses 1 are used at the same time, and each of the sensor apparatuses 1 measures a brainwave signal at its position. The signals are then collected and analyzed by the signal unit 3 so as to be integrated into the single-channel measurement signals.

FIG. 4 is a schematic view showing application of the measurement apparatus according to the present invention. The pumping unit 100 is used to pump gas out of the sleeves of the sensor apparatuses 1 through the communication ports 211, thus allowing the sensor apparatuses 1 to be tightly sucked to the scalp 5 of the subject. The pumping unit 100 can perform the pumping operation through an external device such as a pumping device 4. Through the conductive lines 14, the signal unit 3 collects the signals measured by the sensor apparatuses 1. The signal unit 3 can be an external device, such as a computer or a portable device. Further, the signal unit 3 analyzes the collected signals to obtain variation of the brainwave signals of the subject.

Therefore, the present invention further provides a measurement apparatus, which has: a plurality of sensor apparatuses 1 each comprising: a hollow cylindrical sleeve 11 having a first side 111 and a second side 112 disposed on two end openings thereof, respectively; an electrode 12 disposed on the first side 111 of the sleeve 11 for measuring a signal; a conductive line 14 electrically connected to the electrode 12 for transmitting the signal measured by the electrode 12; and a siphoning portion 13 having a suction port 131 connected and tightly sealed with the second side 112 of the sleeve 11 and an outlet port 132 for discharging gas from the sleeve 11; and a pumping unit 100 including a collection portion 21 having a plurality of communication ports 211 communicating with the outlet ports 132 of the sensor apparatuses 1, respectively, allowing the gas from the sleeves 11 of the sensor apparatuses 1 to be collected in the collection portion 21 and discharged through the pumping unit 100.

The measurement apparatus further has a signal unit 3 connected to the conductive lines 14 of the sensor apparatuses 1 for collecting the signals measured by the electrodes 12 of the sensor apparatuses 1.

Further, the signal unit 3 integrates the signals into single-channel measurement signals.

According to the sensor apparatus of the present invention, gas is pumped out of the sleeve so as to create a vacuum effect to enable the electrode to be sucked to the head of a subject, thus achieving a preferred attachment effect. Also, the present invention overcomes the conventional drawback of discomfort of the subject caused by the use of a conductive material.

In addition, according to the measurement apparatus of the present invention, a plurality of sensor apparatuses can be sucked to the head of the subject to increase the area of measurement coverage, thereby facilitating to obtain complete brainwave signals and increase the analysis reliability.

The above-described descriptions of the detailed embodiments are only to illustrate the preferred implementation according to the present invention, and it is not to limit the scope of the present invention. Accordingly, all modifications and variations completed by those with ordinary skill in the art should fall within the scope of present invention defined by the appended claims.

What is claimed is:

1. A sensor apparatus, comprising:
a hollow cylindrical sleeve having a first side and a second side disposed on two end openings thereof, respectively;
an electrode disposed on the first side of the sleeve for measuring a signal;
a conductive line electrically connected to the electrode for transmitting the signal measured by the electrode; and
a siphoning portion having a suction port connected and tightly sealed with the second side of the sleeve and an outlet port for discharging gas from the sleeve.

2. The sensor apparatus of claim 1, wherein the sleeve is made of silicone.

3. The sensor apparatus of claim 1, wherein the sleeve has a diameter of from 0.5 to 2 cm.

4. The sensor apparatus of claim 1, wherein the signal relates to temperature, pressure or current.

5. The sensor apparatus of claim 1, wherein after the gas is discharged from the sleeve, an internal pressure formed in the sleeve enables the sensor apparatus to be sucked to the skin of a person.

6. A measurement apparatus, comprising:
a plurality of sensor apparatuses each comprising:
a hollow cylindrical sleeve having a first side and a second side disposed on two end openings thereof, respectively;
an electrode disposed on the first side of the sleeve for measuring a signal;
a conductive line electrically connected to the electrode for transmitting the signal measured by the electrode; and
a siphoning portion having a suction port connected and tightly sealed with the second side of the sleeve and an outlet port for discharging gas from the sleeve; and
a pumping unit comprising a collection portion having a plurality of communication ports communicating with the outlet ports of the sensor apparatuses, respectively, allowing the gas from the sleeves of the sensor apparatuses to be collected in the collection portion and discharged through the pumping unit.

7. The measurement apparatus of claim 6, further comprising a signal unit connected to the conductive lines of the sensor apparatuses for collecting the signals measured by the electrodes of the sensor apparatuses.

8. The measurement apparatus of claim 7, wherein the signal unit integrates the signals into single-channel measurement signals.

9. The measurement apparatus of claim 6, wherein at least one of the sleeves is made of silicone.

10. The measurement apparatus of claim 6, wherein at least one of the sleeves has a diameter of from 0.5 to 2 cm.
